(19) United States
(12) Patent Application Publication
(10) Pub. No.: US 2026/0355192 A1
(43) Pub. Date: Nov. 12, 2026

(54) PROPHYLACTIC OR THERAPEUTIC AGENT FOR PERIOSTIN-MEDIATED DISEASE AND PRURITUS ASSOCIATED THEREWITH

(71) Applicants: SAGA UNIVERSITY, Saga-shi, Saga (JP); NATIONAL UNIVERSITY CORPORATION UNIVERSITY OF TOYAMA, Toyama-shi, Toyama (JP)

(72) Inventors: Kenji IZUHARA, Saga (JP); Satoshi NUNOMURA, Saga (JP); Yasuhiro NANRI, Saga (JP); Isao KITAJIMA, Toyama (JP); Daisuke UTA, Toyama (JP)

(73) Assignees: SAGA UNIVERSITY, Saga-shi, Saga (JP); NATIONAL UNIVERSITY CORPORATION UNIVERSITY OF TOYAMA, Toyama-shi, Toyama (JP)

(21) Appl. No.: 18/998,997

(22) PCT Filed: Jul. 27, 2023
(86) PCT No.: PCT/JP2023/027657

§ 371 Date: Apr. 2, 2025

(30) Foreign Application Priority Data
Jul. 28, 2022 (JP) 2022-120377

(51) Int. Cl. A61K 31/506; A61P 17/04

(52) U.S. Cl. CPC A61K 31/506; A61P 17/04

(57) ABSTRACT

The present invention provides a technique for preventing or treating a periostin-mediated disease and pruritus associated with the disease. The present invention is directed to, for example, a prophylactic or therapeutic agent for a periostin-mediated disease or pruritus associated with the disease, which comprises a particular periostin receptor antagonist as an active ingredient.

### TECHNICAL FIELD

The present invention relates to a prophylactic or therapeutic agent for a periostin-mediated disease and pruritus associated with the disease, etc.

### BACKGROUND ART

Atopic dermatitis is a chronic skin disease characterized by recrudescent eczema and intense itching, and the prevalence of atopic dermatitis in young people in the developed nations reaches a little over 10% of the total population. Itching is one of the most unpleasant sensations, and not only reduces concentration, judgment and work efficiency, which are required for activities in daily life, but is also responsible for insomniac symptoms; and hence itching significantly reduces QOL. As therapies for atopic dermatitis, immunosuppressive agents and steroids are externally applied.

For itchy skin symptoms in atopic dermatitis and other diseases, molecular targeted drugs targeting interleukin (IL)-4 and IL-13 receptors and their downstream signaling molecule, Janus kinase (JAK), have been developed in addition to conventionally used antihistamine and antiallergic drugs, and have been found to show a certain level of good therapeutic effect. As also seen in atopic dermatitis, etiological factors resulting in itchy skin symptoms are often complex, and there are still some patients whose itchy skin symptoms are difficult to improve even when treated with the above molecular targeted drugs.

Periostin is an extracellular matrix protein that binds to cell surface receptors belonging to the integrin family and thereby transmits signals into cells, and has been suggested to play a critical role in the amplification and chronicity of allergic skin inflammation (Non-patent Document 1). It has been reported that the local production of periostin is significantly enhanced in the lesions of atopic dermatitis, and the severity of atopic dermatitis correlates with lesional and serum periostin levels. Moreover, periostin has been reported to induce scratching behaviors when administered intracutaneously or subcutaneously to mice, dogs and monkeys (Non-patent Document 2). Further, there has been disclosed a method of treating or alleviating pruritus or chronic pruritus with an integrin αvβ3 antagonist that blocks intracellular signaling generated upon binding between periostin and integrin (Patent Document 1).

Periostin has been reported to mediate the pathology of various diseases. Diseases reported to be mediated by periostin include not only atopic dermatitis and pruritus associated with atopic dermatitis, but also prurigo nodularis, bullous pemphigoid, stasis dermatitis, cutaneous T-cell lymphoma, systemic scleroderma, keloids or hypertrophic scars, allergic rhinitis, chronic liver diseases and chronic kidney diseases, etc., as well as pruritus associated with these diseases (Non-patent Documents 3 to 5).

### PRIOR ART DOCUMENTS

### Patent Documents

Patent Document 1: WO2021/055713

### Non-Patent Documents

Non-patent Document 1: Masuoka M, et al. J Clin Invest. 122:2590-2600, 2012
Non-patent Document 2: Mishra et al. Cell reports, 31:107472, 2020
Non-patent Document 3: Izuhara K, et al. Cell. Mol. Life Sci. 74:4293-4303, 2017
Non-patent Document 4: Hashimoto T, et al. J Invest Dermatol. 141:2338-2343, 2021
Non-patent Document 5: Yang L, Cells. 12, 50, 2023

### SUMMARY OF THE INVENTION

### Problem to be Solved by the Invention

Under these circumstances, there has been a demand for the development of a novel technique for preventing or treating a periostin-mediated disease and pruritus associated with the disease, which is capable of improving the disease and the pruritus.

### Means to Solve the Problem

The present invention has been made in consideration of the above situation, and provides a prophylactic or therapeutic agent for a periostin-mediated disease or pruritus associated with the disease, etc., as shown below.

(1) A prophylactic or therapeutic agent for a periostin-mediated disease or pruritus associated with the disease, which comprises a periostin receptor antagonist as an active ingredient, wherein the antagonist is at least one compound selected from the group consisting of a compound of the following formula (I):

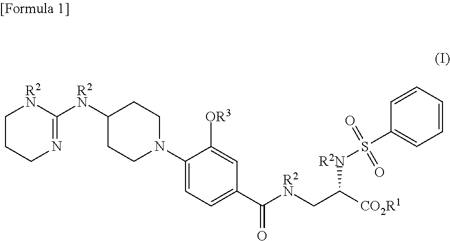

[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid (hereinafter also referred to as “SC-68448”) and (S)-2-(phenylsulfonamido)-3-(4-((S)-3-((1,4,5,6-tetrahydropyrimidin-2-yl)amino)piperidin-1-yl)benzamido)propanoic acid (hereinafter also referred to as “CP derivative 6”), or a prodrug thereof, or a pharmacologically acceptable salt thereof, or a hydrate or solvate thereof.

(2) The prophylactic or therapeutic agent according to (1) above, wherein the compound of formula (I) is (S)-3-(3-methoxy-4-(4-((1,4,5,6-tetrahydropyrimidin-2-yl)amino)piperidin-1-yl)benzamido)-2-(phenylsulfonamido)propanoic acid (hereinafter also referred to as “CP4715”).

(3) The prophylactic or therapeutic agent according to (1) above, wherein the periostin-mediated disease is atopic dermatitis, prurigo nodularis, bullous pemphigoid, stasis dermatitis, cutaneous T-cell lymphoma, systemic scleroderma, a keloid or hypertrophic scar, allergic rhinitis, a chronic liver disease or a chronic kidney disease.

(4) The prophylactic or therapeutic agent according to (1) above, wherein the periostin-mediated disease is atopic dermatitis.

(5) A pharmaceutical composition for preventing or treating a periostin-mediated disease or pruritus associated with the disease, which comprises a periostin receptor antagonist as an active ingredient, wherein the antagonist is at least one compound selected from the group consisting of a compound of the following formula (I):

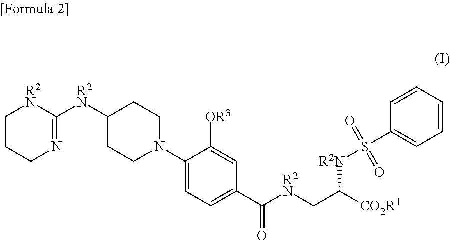

[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid (SC-68448) and (S)-2-(phenylsulfonamido)-3-(4-((S)-3-((1,4,5,6-tetrahydropyrimidin-2-yl)amino)piperidin-1-yl)benzamido)propanoic acid (CP derivative 6), or a prodrug thereof, or a pharmacologically acceptable salt thereof, or a hydrate or solvate thereof.

(6) The pharmaceutical composition according to (5) above, wherein the compound of formula (I) is (S)-3-(3-methoxy-4-(4-((1,4,5,6-tetrahydropyrimidin-2-yl)amino)piperidin-1-yl)benzamido)-2-(phenylsulfonamido)propanoic acid (CP4715).

(7) The pharmaceutical composition according to (5) above, wherein the periostin-mediated disease is atopic dermatitis, prurigo nodularis, bullous pemphigoid, stasis dermatitis, cutaneous T-cell lymphoma, systemic scleroderma, a keloid or hypertrophic scar, allergic rhinitis, a chronic liver disease or a chronic kidney disease.

(8) The pharmaceutical composition according to (5) above, wherein the periostin-mediated disease is atopic dermatitis.

(9) A use of at least one compound selected from the group consisting of a compound of the following formula (I):

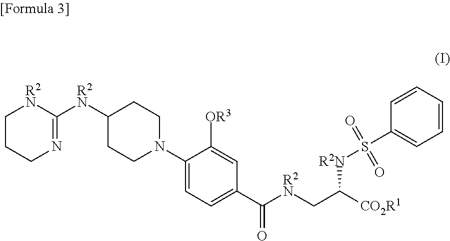

[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid (SC-68448) and (S)-2-(phenylsulfonamido)-3-(4-((S)-3-((1,4,5,6-tetrahydropyrimidin-2-yl)amino)piperidin-1-yl)benzamido)propanoic acid (CP derivative 6), or a prodrug thereof, or a pharmacologically acceptable salt thereof, or a hydrate or solvate thereof for the manufacture of a prophylactic or therapeutic agent for a periostin-mediated disease or pruritus associated with the disease.

(10) The use according to (9) above, wherein the compound of formula (I) is (S)-3-(3-methoxy-4-(4-((1,4,5,6-tetrahydropyrimidin-2-yl)amino)piperidin-1-yl)benzamido)-2-(phenylsulfonamido)propanoic acid (CP4715).

(11) The use according to (9) above, wherein the periostin-mediated disease is atopic dermatitis, prurigo nodularis, bullous pemphigoid, stasis dermatitis, cutaneous T-cell lymphoma, systemic scleroderma, a keloid or hypertrophic scar, allergic rhinitis, a chronic liver disease or a chronic kidney disease.

(12) The use according to (9) above, wherein the periostin-mediated disease is atopic dermatitis.

(13) A method for preventing or treating a periostin-mediated disease or pruritus associated with the disease, which comprises administering an effective amount of a periostin receptor antagonist to a subject in need of treatment, wherein the antagonist is at least one compound selected from the group consisting of a compound of the following formula (I):

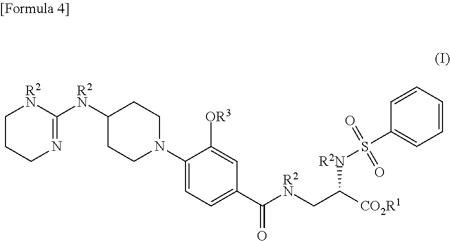

[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid (SC-68448) and (S)-2-(phenylsulfonamido)-3-(4-((S)-3-((1,4,5,6-tetrahydropyrimidin-2-yl)amino)piperidin-1-yl)benzamido)propanoic acid (CP derivative 6), or a prodrug thereof, or a pharmacologically acceptable salt thereof, or a hydrate or solvate thereof.

(14) The method according to (13) above, wherein the compound of formula (I) is (S)-3-(3-methoxy-4-(4-((1,4,5,6-tetrahydropyrimidin-2-yl)amino)piperidin-1-yl)benzamido)-2-(phenylsulfonamido)propanoic acid (CP4715).

(15) The method according to (13) above, wherein the periostin-mediated disease is atopic dermatitis, prurigo nodularis, bullous pemphigoid, stasis dermatitis, cutaneous T-cell lymphoma, systemic scleroderma, a keloid or hypertrophic scar, allergic rhinitis, a chronic liver disease or a chronic kidney disease.

(16) The method according to (13) above, wherein the periostin-mediated disease is atopic dermatitis.

It should be noted that in the inventions shown in (1), (5), (9) and (13) above, the alkyl group intended in R¹, R² and R³ in formula (I) is preferably a lower alkyl group (e.g., an alkyl group containing 1 to 4 carbon atoms), as specifically exemplified by methyl, ethyl, propyl, etc. The aryl group is preferably exemplified by phenyl, tolyl, xylyl, cumenyl, mesyl, naphthyl, biphenyl, etc. The aralkyl group is preferably exemplified by benzyl, phenethyl, etc. It should be noted that these alkyl, aryl and aralkyl groups are not limited in any way, but may each be substituted with a known appropriate substituent(s).

Moreover, a preferred embodiment of the above compound of formula (I) is preferably exemplified by, but not limited to, the compound of the following formula (II):

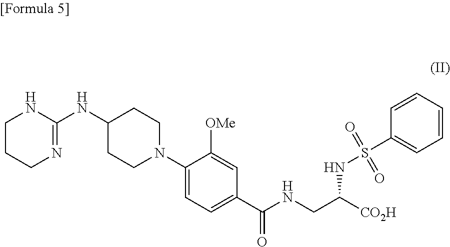 [SMILES: COc1cc(C(=O)NC[C@H](NS(=O)(=O)c2ccccc2)C(=O)O)ccc1N1CCC(NC2=NCCCN2)CC1]

(i.e., (S)-3-(3-methoxy-4-(4-((1,4,5,6-tetrahydropyrimidin-2-yl)amino)piperidin-1-yl)benzamido)-2-(phenylsulfonamido)propanoic acid (CP4715)).

### Effects of the Invention

The present invention enables the provision of not only a prophylactic or therapeutic agent for a periostin-mediated disease and pruritus associated with the disease, which is effective in the improvement of the disease and the pruritus, but also a pharmaceutical composition for preventing or treating the disease and the pruritus, and a method for preventing or treating the disease and the pruritus, etc.

### BRIEF DESCRIPTION OF THE DRAWINGS

FIG. 1 contains graphs showing the suppressive effect against scratching behaviors in Facial Atopic Dermatitis with Scratching (FADS) mice (see Example 1 described later) upon repeated systemic administration of CP4715.

FIG. 2 is a graph showing the suppressive effect against scratching behaviors in FADS mice upon single systemic administration of CP4715.

FIG. 3 is a graph showing the suppressive effect of CP4715 against spontaneous firing recorded from neurons in the posterior horn surface of the spinal cord in FADS mice.

FIG. 4 is a graph showing the suppressive effect against scratching behaviors in FADS mice upon single systemic administration of various compounds.

FIG. 5 is a graph showing the suppressive effect against scratching behaviors in FADS mice upon single systemic administration of GLPG-0187.

FIG. 6 is a graph showing the inhibitory effect of various compounds against periostin-dependent cell adhesion.

FIG. 7 is a graph showing the suppressive effect against scratching behaviors in FADS mice upon repeated percutaneous administration of CP4715.

### DESCRIPTION OF EMBODIMENTS

The present invention will be described in more detail below. The scope of the present invention is not limited by the following descriptions, and any embodiments other than those illustrated below may also be carried out with appropriate modifications without departing from the spirit of the present invention. It should be noted that this specification incorporates the specification of Japanese Patent Application No. 2022-120377 (filed on Jul. 28, 2022) in its entirety, based on which the present application claims priority. Moreover, all publications cited herein, including prior art documents, patent gazettes and other patent documents, are incorporated herein by reference.

The prophylactic or therapeutic agent for a periostin-mediated disease and pruritus (e.g., itchy skin symptoms) associated with the disease according to the present invention (hereinafter also referred to as “the prophylactic or therapeutic agent of the present invention”), and the pharmaceutical composition for preventing or treating a periostin-mediated disease and pruritus (e.g., itchy skin symptoms) associated with the disease according to the present invention (hereinafter also referred to as “the pharmaceutical composition of the present invention”) are characterized by comprising, as an active ingredient, at least one compound (hereinafter also referred to as “the compound of the present invention”) selected from the group consisting of a compound of the following formula (I):

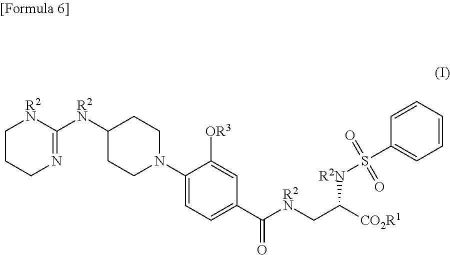

[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid (SC-68448) and (S)-2-(phenylsulfonamido)-3-(4-((S)-3-((1,4,5,6-tetrahydropyrimidin-2-yl)amino)piperidin-1-yl)benzamido)propanoic acid (CP derivative 6), or a prodrug thereof, or a pharmacologically acceptable salt thereof, or a hydrate or solvate thereof (the compound of the present invention and the above prodrug, salt, hydrate and solvate are also collectively referred to as “the compound of the present invention or other form thereof”), as described above.

In the above formula (I), the alkyl group intended in R¹, R² and R³ is preferably a lower alkyl group (e.g., an alkyl group containing 1 to 4 carbon atoms), as specifically exemplified by methyl, ethyl, propyl, etc. The aryl group is preferably exemplified by phenyl, tolyl, xylyl, cumenyl, mesyl, naphthyl, biphenyl, etc. The aralkyl group is preferably exemplified by benzyl, phenethyl, etc. It should be noted that these alkyl, aryl and aralkyl groups are not limited in any way, but may each be substituted with a known appropriate substituent(s).

In the present invention, a preferred embodiment of the above compound of formula (I) is preferably exemplified by, but not limited to, the compound of the following formula (II):

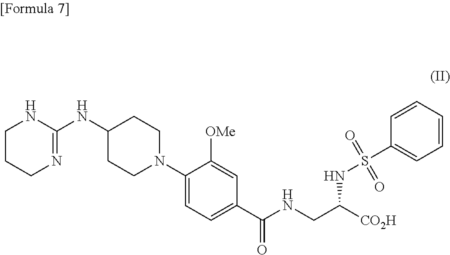 [SMILES: COc1cc(C(=O)NC[C@H](NS(=O)(=O)c2ccccc2)C(=O)O)ccc1N1CCC(NC2=NCCCN2)CC1]

i.e., (S)-3-(3-methoxy-4-(4-((1,4,5,6-tetrahydropyrimidin-2-yl)amino)piperidin-1-yl)benzamido)-2-(phenylsulfonamido)propanoic acid. The above compound of formula (II) is known as “CP4715” (Nanri Y, et al. Am J Respir Cell Mol Biol. 62:204-216, 2020), and its CAS registration number is 247034-73-7 (free form).

3-(3,5-Dichlorophenyl)-3-(2-(3-guanidinobenzamido)acetamido)propanoic acid is known as “SC-68448” (Carron C P, et al. Cancer Res. 58:1930-1935, 1998), and is a compound having the chemical structure of the following formula (III). The CAS registration number of SC-68448 is 188804-07-1 (free form).

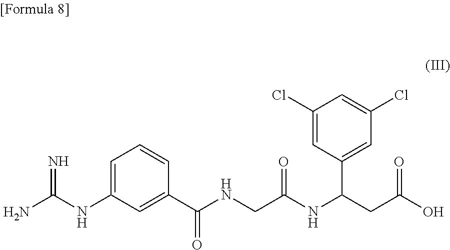 [SMILES: N=C(N)Nc1cccc(C(=O)NCC(=O)NC(CC(=O)O)c2cc(Cl)cc(Cl)c2)c1]

(S)-2-(Phenylsulfonamido)-3-(4-((S)-3-((1,4,5,6-tetrahydropyrimidin-2-yl)amino)piperidin-1-yl)benzamido)propanoic acid (CP derivative 6) is a known compound (Nanri Y, et al. Am J Respir Cell Mol Biol. 62:204-216, 2020), and has the chemical structure of the following formula (IV). “CP derivative 6” and CP4715 have a common fundamental structure. The CAS registration number of CP derivative 6 is 334618-85-8 (free form).

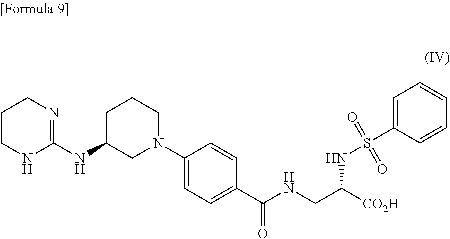 [SMILES: O=C(NC[C@H](NS(=O)(=O)c1ccccc1)C(=O)O)c1ccc(N2CCC[C@H](NC3=NCCCN3)C2)cc1]

“CP4715,” “SC-68448” and “CP derivative 6” used here may be commercially available products, but are not limited thereto, and may also be synthesized, extracted and purified independently for this purpose. “CP4715” is a compound having an antagonistic effect on both glycoprotein (GP) IIb/IIIa receptor playing a major role in thrombus formation and αvβ3 integrin (cell adhesion molecule) involved in leukocyte adhesion, etc., and has been known as an active ingredient in therapeutic agents for circulatory system diseases (antithrombotic agents). In this regard, αvβ3 integrin is a receptor for periostin, fibronectin and vitronectin, etc., and periostin has been known to activate sensory nerves through αvβ3 integrin (see Non-patent Document 2 listed above). SC-68448 serves as an integrin αvβ3 antagonist and has been reported to inhibit binding between integrin αvβ3 and vitronectin (Carron C P, et al. Cancer Res. 58:1930-1935, 1998). CP derivative 6 has been reported to inhibit binding between integrin αvβ3 and periostin or vitronectin (Nanri Y, et al. Am J Respir Cell Mol Biol. 62:204-216, 2020).

It should be noted that the present invention may encompass the following embodiments in addition to the prophylactic or therapeutic agent of the present invention and the pharmaceutical composition of the present invention described above:
(i) a method for preventing or treating a periostin-mediated disease or pruritus associated with the disease, which comprises using the compound of the present invention or other form thereof, more particularly, for example, administering an effective amount of the compound of the present invention or other form thereof to a subject in need of treatment (i.e., a patient with or at risk of a periostin-mediated disease or pruritus (e.g., itchy skin symptoms) associated with the disease, or a non-human mammal with or at risk of the disease or the pruritus);
(ii) the use of the compound of the present invention or other form thereof for the manufacture of a prophylactic or therapeutic agent for a periostin-mediated disease or pruritus (e.g., itchy skin symptoms) associated with the disease;
(iii) the use of the compound of the present invention or other form thereof for preventing or treating a periostin-mediated disease or pruritus (e.g., itchy skin symptoms) associated with the disease; and
(iv) the compound of the present invention or other form thereof for preventing or treating a periostin-mediated disease or pruritus (e.g., itchy skin symptoms) associated with the disease.

In the present invention, the term “periostin-mediated disease” refers to a disease in which periostin is involved in the onset or progression of the disease. The term “periostin-mediated” is intended to mean a state where periostin directly acts on somatic cells or somatic tissues, e.g., periostin binds to its receptor to thereby activate intracellular signaling systems, whereby periostin is involved in the onset or progression of the disease. Further, the term “periostin-mediated” is also intended to mean a state where periostin acts on somatic tissues or somatic cells to thereby newly produce, express or release or activate a biomolecule, and this biomolecule causes the onset or progression of the disease.

Examples of a periostin-mediated disease include, but are not limited to, atopic dermatitis, prurigo nodularis, bullous pemphigoid, stasis dermatitis, cutaneous T-cell lymphoma, systemic scleroderma, keloids or hypertrophic scars, allergic rhinitis, chronic liver diseases and chronic kidney diseases, etc., as well as pruritus associated with these diseases.

Atopic dermatitis is a chronic skin disease whose major lesion is pruritic eczema with repeated exacerbation and remission. In patients with atopic dermatitis, it has been reported that their serum periostin levels are higher than those of normal people, and the local production of periostin is significantly enhanced in their lesions, and that the severity of atopic dermatitis correlates with lesional and serum periostin levels (Non-patent Documents 1 and 2 listed above).

Scratching associated with pruritus causes a vicious cycle of exacerbating skin symptoms and further enhancing pruritus (itch-scratch cycle) and has been known to be involved in the pathogenesis of atopic dermatitis (Tominaga M, Takamori K. Allergol Int. (2022) 71:265-277). IL-31, a cytokine produced primarily from Th2 cells, is known as one of the major itching inducers in atopic dermatitis (Datsi A, et al. Allergy. 76:2982-2997, 2021), and has been reported to bind to the IL-31 receptor and thereby transmit pruritic signals to the central nervous system (Mollanazar N K, et al. Clin Rev Allergy Immunol. 51:263-292, 2020). In a clinical trial where an anti-IL-31 receptor antibody (nemolizumab) was administered to patients with atopic dermatitis, nemolizumab was found not only to improve pruritus but also to show an improving effect on skin inflammation (Kabashima K, et al. N Engl J Med. 383:141-150, 2020). Namely, this indicates that drugs for alleviating pruritus associated with atopic dermatitis are also useful as prophylactic or therapeutic agents for atopic dermatitis.

Prurigo nodularis is a papular skin disease accompanied by intense itching, and the intensity of periostin immunostaining in the dermis has been reported to correlate with the intensity of itching in prurigo nodularis (Hashimoto T, et al. Acta Derm Venereol. 101:adv00375, 2021).

Bullous pemphigoid is an autoimmune blistering disease with Th2 immunity, which is a skin disease accompanied by moderate-to-severe itching. The intensity of itching in bullous pemphigoid directly correlates with the dermal deposition of periostin (Hashimoto T, et al. J Am Acad Dermatol. 83:53-62, 2020), thus suggesting a possibility that periostin may be directly involved in bullous pemphigoid-associated itching.

Stasis dermatitis is a disease that typically causes inflammation in the lower leg skin of elderly people due to chronic venous insufficiency and lymphedema, etc. It has been reported that periostin may contribute to stasis dermatitis-associated itching through the promotion of IL-31 production by macrophages (Hashimoto T, et al. J Invest Dermatol. 140:850-859, 2020).

Cutaneous T-cell lymphoma is a group of malignant lymphomas occurring in the skin, whose tumor-originating cells are T cells. In cutaneous T-cell lymphoma, the prevalence of itching is high, but the expression of periostin is increased in lesional skin samples of cutaneous T-cell lymphoma (Furudate S, et al. Exp Dermatol. 25:107-112, 2016), thus suggesting a possibility that periostin may partially contribute to itching in cutaneous T-cell lymphoma.

Systemic scleroderma and keloids or hypertrophic scars are diseases that commonly share an abnormal dermal fibrosis and are highly accompanied by pruritus. In lesional skin samples from systemic scleroderma patients and keloid patients, the enhanced expression of dermal periostin has been observed (Maeda D, et al. J Plast Surg Hand Surg. 53:288-294, 2019). In addition, it has been reported that serum periostin levels are elevated and correlate with disease severity in patients with systemic scleroderma (Yamaguchi Y, et al. Br J Dermatol. 168:717-725, 2013). These findings suggest a possibility that periostin may be involved in pruritus associated with systemic scleroderma and keloids or hypertrophic scars.

Allergic rhinitis is often accompanied by itching, and it has been confirmed that periostin is highly expressed in the nasal mucosa of patients with allergic rhinitis (Ishida A, et al. Allergol Int, 61:589-595, 2012). For this reason, there is a possibility that periostin may partially contribute to itching in allergic rhinitis (Ohta N, et al. Allergol Int. 63:171-180, 2014).

Periostin has been reported to inhibit the expression of peroxisome proliferator-activated receptor-α and thereby induce liver fibrosis (J Gastroenterol Hepatol. 35:2140-2150, 2020). Moreover, chronic liver diseases such as cirrhosis may cause intractable itching which is less responsive to even the administration of antihistamine drugs, and periostin is deemed to be involved in these diseases.

The involvement of periostin has also been reported in chronic kidney diseases. The expression levels of periostin in the kidney and urine have been shown to highly correlate with the stage of the pathology and the decline of renal function (Prakoura N, et al. Cell Mol Life Sci. 74:4315-4320, 2017, Jia Y-Y, et al. Ren Fail. 42:1166-1172, 2020). The decline of renal function results in the need for artificial dialysis. Patients undergoing artificial dialysis have skin dryness, which is responsible for further causing pruritus (Morton C A, et al. Nephrol Dial Transplant. 10:2031-2036, 1996). When skin dryness progresses, the skin barrier function is reduced and thus nerve fibers (C-fibers) are elongated into the epidermis (Andoh T, et al. Neurosci Lett. 672:84-89, 2018), and there would be a possibility that itching may be caused when the periostin receptor present in the nerve fibers is activated with periostin (Non-patent Document 2 listed above).

As discussed above, various human diseases have been known to be mediated by periostin. However, there is no report showing that a therapeutic effect or a prophylactic effect was actually obtained when periostin receptor antagonists were administered against these human diseases.

In the present invention, pruritus may be exemplified by itchy skin symptoms. Examples of itchy skin symptoms include, but are not limited to, those arising from skin diseases or inflammation. Examples of skin diseases or inflammation include, but are not limited to, atopic dermatitis, contact dermatitis, and prurigo, etc.

In the present invention, the terms “prevent(ing)” and “treat(ing)” are intended to also mean suppressing and/or inhibiting or alleviating a periostin-mediated disease and pruritus (e.g., itchy skin symptoms) associated with the disease. Thus, the present invention may include a suppressor, inhibitor or alleviator for symptoms of a periostin-mediated disease and pruritus (e.g., itchy skin symptoms) associated with the disease, which comprises the compound of the present invention or other form thereof, a pharmaceutical composition for suppressing, inhibiting or alleviating the symptoms, which comprises the compound of the present invention or other form thereof, a method for suppressing, inhibiting or alleviating the symptoms, which comprises administering the compound of the present invention or other form thereof to a subject, as well as the use of the compound of the present invention or other form thereof in the manufacture of a drug for suppressing, inhibiting or alleviating the symptoms. Moreover, the treatment of a periostin-mediated disease and pruritus (e.g., itchy skin symptoms) includes, for example, suppression of symptom progression, improvement of prognosis and treatment leading to remission, etc., and further includes prevention of symptoms and prevention of recurrence, etc., without being limited to the common meaning of treatment.

As an active ingredient in the prophylactic or therapeutic agent of the present invention and the pharmaceutical composition of the present invention, a derivative of the compound of the present invention may be used in combination with or in place of the compound of the present invention. Such a derivative is not limited in any way as long as it is considered to be the intended derivative on the basis of common knowledge shared among those skilled in the art, e.g., in terms of having a chemical structure derived from the compound of the present invention. However, preferred is a derivative having a prophylactic or therapeutic effect on a periostin-mediated disease and pruritus (e.g., itchy skin symptoms) associated with the disease at the same level as the compound of the present invention.

Examples of the compound of the present invention or a derivative thereof include not only those which undergo in vivo metabolism such as oxidation, reduction, hydrolysis or conjugation, but also compounds which produce the compound of the present invention or a derivative thereof upon in vivo metabolism such as oxidation, reduction or hydrolysis (i.e., so-called prodrugs). In the present invention, such a prodrug refers to a compound prepared from its parent compound by modification with a pharmacologically acceptable group which is commonly used in prodrugs, as exemplified by a compound which is provided with properties such as improved chemical stability, effect sustainability or in vivo absorbability, and can be expected to exert the intended effect when converted into its parent compound in the intestinal tract or skin tissue, etc. For example, a prodrug of the compound of the present invention can be prepared in a standard manner by using a prodrug-forming reagent such as a corresponding halide to introduce a prodrug-constituting group(s) as appropriate in a standard manner into any one or more groups selected from among the groups in this compound, which can be used for prodrug formation (e.g., a hydroxyl group, an amino group, a carboxyl group and other groups), optionally followed by isolation and purification. As intended here, the above prodrug-constituting groups preferably include, but are not limited to, lower alkyl-CO—, lower alkyl-O-lower alkylene-CO—, lower alkyl-OCO-lower alkylene-CO—, lower alkyl-OCO—, and lower alkyl-O-lower alkylene-OCO—, etc.

As an active ingredient in the prophylactic or therapeutic agent of the present invention and the pharmaceutical composition of the present invention, a pharmacologically acceptable salt of the compound of the present invention and/or a derivative thereof or a prodrug thereof may be used in combination with or in place of the compound of the present invention and/or a derivative thereof or a prodrug thereof.

Such a pharmacologically acceptable salt of the compound of the present invention and/or a derivative thereof is not limited in any way, but preferred examples include halogenated hydroacid salts (e.g., hydrochloride, hydrobromide, and hydroiodide), inorganic acid salts (e.g., sulfate, nitrate, perchlorate, phosphate, carbonate, and bicarbonate), organic carboxylic acid salts (e.g., acetate, trifluoroacetate, maleate, tartrate, fumarate, and citrate), organic sulfonic acid salts (e.g., methanesulfonate, trifluoromethanesulfonate, ethanesulfonate, benzenesulfonate, toluenesulfonate, and camphorsulfonate), amino acid salts (e.g., aspartate, and glutamate), quaternary amine salts, alkali metal salts (e.g., sodium salt, and potassium salt), alkaline earth metal salts (e.g., magnesium salt, and calcium salt).

The above compound of the present invention or other form thereof for use as an active ingredient encompasses all isomers possible in terms of the compound's structure (e.g., geometrical isomers, optical isomers based on asymmetric carbons, rotational isomers, stereoisomers, and tautomers) and mixtures of two or more of these isomers, and is not limited to the descriptions about the structural formula shown for convenience’ sake. Moreover, the compound of the present invention or other form thereof may be in S-configuration, R-configuration or RS-configuration, and is not limited in any way. Further, the compound of the present invention or other form thereof may be present in the form of a hydrate or solvate, depending on its type. In the present invention, such a hydrate or solvate also falls within the compound of the present invention or other form thereof, and may be used as an active ingredient in the suppressor or inhibitor of the present invention and the pharmaceutical composition of the present invention. Such a solvate is not limited in any way, but is exemplified by a solvate with ethanol, etc.

In the prophylactic or therapeutic agent of the present invention and the pharmaceutical composition of the present invention, the content of the compound of the present invention or other form thereof as an active ingredient is not limited in any way and may be determined as appropriate. For example, the content may be set within the range of 0.01% to 99% by weight, relative to the total weight of the prophylactic or therapeutic agent or the pharmaceutical composition, and preferably set within the range of 0.01% to 30% by weight, more preferably 0.05% to 20% by weight, even more preferably 0.1% to 10% by weight, relative to the total weight of the prophylactic or therapeutic agent or the pharmaceutical composition. The content of the active ingredient within the above range is sufficient for the prophylactic or therapeutic agent of the present invention and the pharmaceutical composition of the present invention to exert a prophylactic or therapeutic effect on a periostin-mediated disease and pruritus (e.g., itchy skin symptoms) associated with the disease.

In addition to the compound of the present invention or other form thereof, the prophylactic or therapeutic agent of the present invention and the pharmaceutical composition of the present invention may further comprise other ingredients as long as the effect of the present invention is not significantly impaired. Other ingredients include drugs already known or under development, as exemplified by a moisturizer, a steroid drug, an immunosuppressive agent, an antihistamine drug, a JAK inhibitor, and an IL-4/IL-13 inhibitor, etc., which may be used either alone or in combination. Further, the suppressor or inhibitor of the present invention and the pharmaceutical composition of the present invention may also comprise, e.g., ingredients commonly used for drug preparation as described later.

The prophylactic or therapeutic agent of the present invention and the pharmaceutical composition of the present invention may be administered to a subject, i.e., a human or a non-human mammal (e.g., rat, rabbit, sheep, pig, cow, cat, dog, monkey) by various routes of administration, as specifically exemplified by oral administration or parenteral administration (e.g., intravenous injection, intramuscular injection, intraperitoneal injection, subcutaneous injection, intrarectal administration, percutaneous administration and external application). Thus, the compound of the present invention or other form thereof may be used alone, but may also be formulated with a pharmaceutically acceptable carrier into an appropriate dosage form in a manner commonly used, depending on the intended route of administration.

Dosage forms for oral formulations may be exemplified by tablets, powders, fine granules, granules, coated tablets, capsules, solutions for internal use, suspensions, emulsions, syrups, and troches, etc., while dosage forms for parenteral formulations may be exemplified by injections (including drops), inhalants, ointments, creams, nose drops, and liposomes, etc. It should be noted that when formulated into various oral formulations as mentioned above, the prophylactic or therapeutic agent of the present invention and the pharmaceutical composition of the present invention may optionally be used as supplements (e.g., falling within functional food products). It should be noted that each dosage form may be prepared according to the procedures described in the Japanese Pharmacopoeia.

Examples of carriers which may be used to formulate these formulations include commonly used excipients, binders, disintegrants, lubricants, colorants, and correctives, as well as optionally stabilizers, emulsifiers, absorbefacients, surfactants, pH adjusters, antiseptics, antioxidants, extenders, humectants, surface active agents, dispersants, buffering agents, preservatives, solvent aids, oleaginous bases, emulsifying bases, aqueous bases, suspending bases and soothing agents, etc., which may be blended with known ingredients available for use as source materials for pharmaceutical formulations and then formulated in a standard manner.

Non-toxic ingredients available for this purpose may be exemplified by animal and vegetable oils such as soybean oil, beef tallow, and synthetic glycerides; hydrocarbons such as liquid paraffin, squalane, petrolatum, plastibase and hard paraffin; ester oils such as octyldodecyl myristate, and isopropyl myristate; higher alcohols such as cetostearyl alcohol, and behenyl alcohol; silicone resin; silicone oil; surfactants such as polyoxyethylene fatty acid esters, sorbitan fatty acid esters, glycerin fatty acid esters, polyoxyethylene sorbitan fatty acid esters, polyoxyethylene hydrogenated castor oil, and polyoxyethylene-polyoxypropylene block copolymers; water-soluble polymers such as hydroxyethylcellulose, polyacrylic acid, carboxyvinyl polymers, polyethylene glycol, polyvinylpyrrolidone, and methylcellulose; lower alcohols such as ethanol, and isopropanol; polyhydric alcohols (polyols) such as glycerin, propylene glycol, dipropylene glycol, sorbitol, and polyethylene glycol; sugars such as glucose, and sucrose; inorganic powders such as silicic anhydride, magnesium aluminum silicate, and aluminum silicate; inorganic salts such as sodium chloride, and sodium phosphate; and purified water. These ingredients may be in the form of salts or hydrates thereof.

Preferred examples of excipients include lactose, fructose, corn starch, sucrose, glucose, mannitol, sorbit, crystalline cellulose, and silicon dioxide, etc. Preferred examples of binders include polyvinyl alcohol, polyvinyl ether, methylcellulose, ethylcellulose, gum arabic, tragacanth, gelatin, shellac, hydroxypropylmethylcellulose (hypromellose), hydroxypropylcellulose, polyvinylpyrrolidone, polypropylene glycol-polyoxyethylene block polymers, and meglumine, etc. Preferred examples of disintegrants include starch, agar, gelatin powder, crystalline cellulose, calcium carbonate, sodium bicarbonate, calcium citrate, dextrin, pectin, carboxymethylcellulose (carmellose) and carboxymethylcellulose calcium (carmellose calcium), etc. Preferred examples of lubricants include magnesium stearate, talc, polyethylene glycol, silica, and hydrogenated vegetable oils, etc. Preferred examples of colorants include those approved for addition to pharmaceutical products. Preferred examples of correctives include cocoa powder, menthol, aromatic powder, peppermint oil, borneol, and cinnamon powder, etc. These ingredients may be in the form of salts or hydrates thereof.

Moreover, pH adjusters are used when the prophylactic or therapeutic agent of the present invention and the pharmaceutical composition of the present invention are formulated as injections, etc. pH adjusters are not limited in any way as long as they are medically, pharmacologically (pharmaceutically) or physiologically acceptable, and specific examples include alkali metal hydroxides such as sodium carbonate, potassium carbonate, sodium ethoxide, potassium butoxide, sodium hydroxide and potassium hydroxide; alkali metal hydrides such as sodium hydride and potassium hydride; alkali metal or alkaline earth metal carbonates; and alkali metal alkoxides, etc. pH adjusters to be incorporated may be used either alone or in any combination. When formulated as injections, etc., the prophylactic or therapeutic agent of the present invention and the pharmaceutical composition of the present invention may be adjusted as appropriate with a pH adjuster(s) to an appropriate pH. In the present invention, pH is preferably 3 to 6, more preferably 3 to 5, even more preferably 3 to 4.5, and particularly preferably 3.5 to 4.5.

It should be noted that when formulated as injections, the prophylactic or therapeutic agent of the present invention and the pharmaceutical composition of the present invention may contain cyclodextrins. Cyclodextrins are not limited in any way as long as they are medically, pharmacologically (pharmaceutically) or physiologically acceptable. Such cyclodextrins may be exemplified by unmodified cyclodextrins, modified cyclodextrins, etc. Examples of unmodified cyclodextrins include α-cyclodextrin, β-cyclodextrin, and γ-cyclodextrin, etc. Likewise, examples of modified cyclodextrins include alkylated cyclodextrins (e.g., dimethyl-α-cyclodextrin, dimethyl-β-cyclodextrin, dimethyl-γ-cyclodextrin), hydroxyalkylated cyclodextrins (e.g., hydroxypropyl-α-cyclodextrin, hydroxypropyl-β-cyclodextrin, hydroxypropyl-γ-cyclodextrin), sulfoalkyl ether cyclodextrins (e.g., sulfobutyl ether-α-cyclodextrin, sulfobutyl ether-β-cyclodextrin, sulfobutyl ether-γ-cyclodextrin), and branched cyclodextrins (e.g., maltosyl-α-cyclodextrin, maltosyl-β-cyclodextrin, maltosyl-γ-cyclodextrin), etc.

Cyclodextrins may be used either alone or in any combination. A preferred cyclodextrin to be incorporated is hydroxypropyl-β-cyclodextrin or sulfobutyl ether-β-cyclodextrin, and more preferred is sulfobutyl ether-β-cyclodextrin.

The content of cyclodextrins is not limited in any way and may be determined as appropriate depending on their type, the purpose of and how to use the injection, etc. For example, the content of cyclodextrins is preferably 5% to 50% by weight based on the total weight of the injection, and is more preferably 10% to 40% by weight, even more preferably 10% to 30% by weight, based on the total weight of the injection.

The dose of the prophylactic or therapeutic agent of the present invention and the pharmaceutical composition of the present invention may generally be determined extensively as appropriate for the age and body weight of a subject (patient) to be administered, the type and progression of disease, as well as the route of administration, the frequency of administration (per day) and the period of administration, etc., in consideration of the ratio of the active ingredient (the compound of the present invention or other form thereof) incorporated into the formulation.

A detailed explanation will be given below for the case where the prophylactic or therapeutic agent of the present invention and the pharmaceutical composition of the present invention are used as parenteral formulations or oral formulations.

For use as parenteral formulations, the prophylactic or therapeutic agent of the present invention and the pharmaceutical composition of the present invention may usually be formulated into any dosage form. In the case of various types of injections, for example, they may be provided in the form of unit dose ampules or multi-dose containers or as freeze-dried powders which are dissolved again in a diluent before use. Such a parenteral formulation may comprise not only the compound of the present invention or other form thereof serving as an active ingredient, but also various known excipients and/or additives as appropriate for each dosage form as long as the effect of the above active ingredient is not impaired. Examples of excipients and/or additives include water, glycerol, propylene glycol, or aliphatic polyalcohols such as polyethylene glycol, etc., in the case of various types of injections.

The dose (daily dose) of such a parenteral formulation is not limited in any way. For example, in the case of various types of injections, the dose may generally be set such that the compound of the present invention or other form thereof serving as an active ingredient can be taken in an amount of 0.01 to 1000 mg, 0.05 to 500 mg or 0.1 to 50 mg, per kg body weight of a subject to be applied (e.g., a test subject, a patient), or alternatively, can be taken in an amount of 0.5 to 20 mg or can be taken in an amount of 1 to 10 mg.

For use as oral formulations, the prophylactic or therapeutic agent of the present invention and the pharmaceutical composition of the present invention may usually be formulated into any dosage form among those mentioned above, or alternatively, may be formulated into freeze-dried products which are dissolved again before use. Such an oral formulation may comprise not only the compound of the present invention or other form thereof serving as an active ingredient, but also various known excipients and/or additives as appropriate for each dosage form as long as the effect of the above active ingredient is not impaired. Examples of excipients and/or additives include binders (e.g., syrup, gum arabic, gelatin, sorbitol, tragacanth, polyvinylpyrrolidone), fillers (e.g., lactose, sugar, corn starch, potato starch, calcium phosphate, sorbitol, glycine), lubricants (e.g., magnesium stearate, talc, polyethylene glycol, silica), disintegrants (e.g., various types of starches), and wetting agents (e.g., sodium lauryl sulfate), etc.

The dose (daily dose) of such an oral formulation may generally be set such that the compound of the present invention or other form thereof serving as an active ingredient can be taken in an amount of 0.05 to 5000 mg, 0.1 to 1000 mg or 0.1 to 100 mg, per kg body weight of a subject to be applied (e.g., a test subject, a patient), or alternatively, can be taken in an amount of 0.5 to 50 mg or can be taken in an amount of 1 to 10 mg. Moreover, the ratio of the active ingredient incorporated into the oral formulation is not limited in any way and may be set as appropriate in consideration of the frequency of administration per day, etc.

It should be noted that in the present invention, for prevention or treatment of a periostin-mediated disease and pruritus (e.g., itchy skin symptoms) associated with the disease, it is also possible to use a kit comprising the compound of the present invention or other form thereof (as specifically exemplified by a kit comprising the prophylactic or therapeutic agent of the present invention or the pharmaceutical composition of the present invention mentioned above).

In such a kit, the compound of the present invention or other form thereof may be in any form, but may be provided, for example, in a dissolved state in consideration of its stability (storage quality) and easiness of use, etc. Such a kit may also comprise other constituent elements, as appropriate, in addition to the compound of the present invention or other form thereof.

The kit is required to comprise at least the compound of the present invention or other form thereof as a constituent element. Thus, the kit may be configured to comprise all the constituent elements essential for prevention or treatment of a periostin-mediated disease and pruritus (e.g., itchy skin symptoms) associated with the disease, either together with or separately from the compound of the present invention or other form thereof, without being limited thereto.

### EXAMPLES

The present invention will be further described in more detail by way of the following examples, although the present invention is not limited only to these examples.

### Example 1

Preparation of Facial Atopic Dermatitis with Scratching (FADS) Mice

Nestin-cre transgenic mice were crossed with IKK2^{ff} mice to prepare Nestin-cre/Ikk2^{ff} mice specifically lacking IKK2 in their facial skin fibroblasts (Nunomura S, et al. J Invest Dermatol. 139:1274-1283, 2019). These mice inevitably develop atopic dermatitis-like rash limited to the face and intense itchy symptoms, and were therefore designated as Facial Atopic Dermatitis with Scratching (FADS) mice. FADS mice show significantly increased serum periostin when compared to control mice and also show high expression of periostin in the dermis of the facial skin; upon disruption of the periostin gene in FADS mice, their scratching behaviors are significantly suppressed when compared to control mice; and scratching behaviors in FADS mice are suppressed upon intraperitoneal administration of an anti-periostin monoclonal antibody which neutralizes periostin, thus indicating that scratching behaviors in FADS mice are periostin-mediated (Nunomura S, et al. Cell Rep. 42:111933, 2023). For evaluation of the prophylactic or therapeutic agent of the present invention, FADS mice at 3 to 4 weeks of age or older were used because of showing spontaneous scratching behaviors. It should be noted that in this Example section, the above compound of formula (I) (more particularly “CP4715”) was used as the prophylactic or therapeutic agent of the present invention.

### Inhibition of Scratching Behaviors in FADS Mice Upon Repeated Systemic Administration of CP4715

FADS mice were acclimated for 20 minutes to the environment of a recording cage, and then recorded with a video camera. The recorded videos were replayed, and scratching behaviors were counted assuming that hind paw scratching to the face and the subsequent movement of toe licking or paw lowering to the ground were regarded as one cycle of scratching behavior.

CP4715 was dissolved at 100 mg/mL in a diluent (physiological saline containing 50% dimethyl sulfoxide (DMSO) and 89.5 mM hydrochloric acid), from which an amount corresponding to 107.4 μmol/kg per mouse was then taken and diluted with 0.2 mL of PBS, and then administered twice a day under the dorsal skin. Mice administered with 0.2 mL of PBS containing an equal amount of the diluent as calculated based on the body weight were used as a control group.

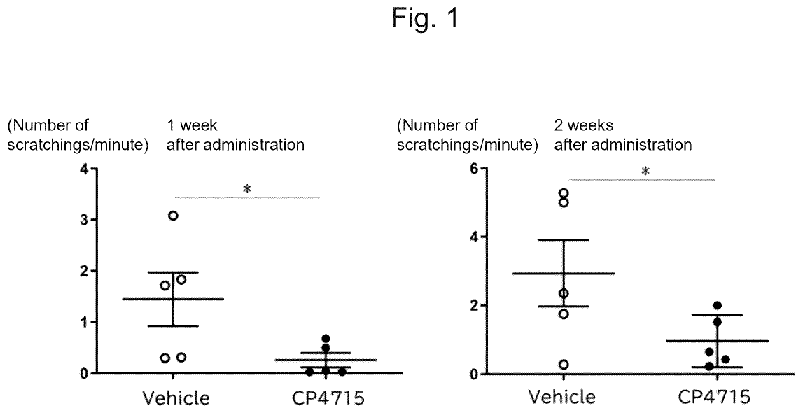

At 1 week and 2 weeks after drug administration, the mice were recorded for 60 minutes and analyzed to calculate the number of scratchings per minute for evaluation. For statistical analysis, Mann-Whitney's U-test was used. A P-value of less than 0.05 was considered to be statistically significant. The results obtained are shown in FIG. 1. As shown in FIG. 1, scratching behaviors at 1 week and 2 weeks after CP4715 administration were significantly suppressed (P<0.05).

### Inhibition of Scratching Behaviors in FADS Mice Upon Single Systemic Administration of CP4715

Prior to CP4715 administration, FADS mice were recorded for 60 minutes and then allowed to rest for 30 minutes. Then, CP4715 was dissolved at 89.5 mM in a diluent (physiological saline containing 50% DMSO and 89.5 mM HCL), from which an amount corresponding to 53.7 μmol/kg per mouse was then taken and diluted with 0.3 mL of PBS, and then administered intraperitoneally. Mice administered with 0.3 mL of PBS containing an equal amount of the diluent as calculated based on the body weight were used as a control group.

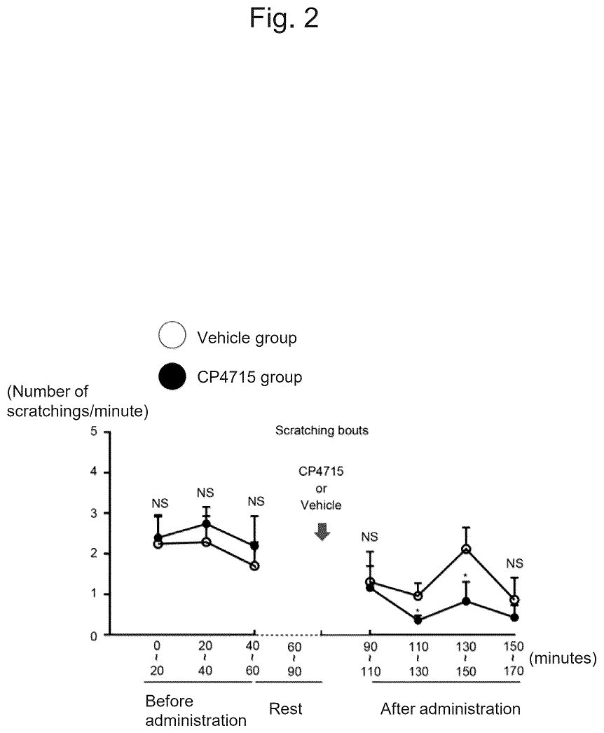

The mice were recorded for 80 minutes immediately after drug administration. The number of scratchings was determined every 20 minutes both before and after administration to calculate the number scratchings per minute for evaluation. For statistical analysis, Mann-Whitney's U-test was used. A P-value of less than 0.05 was considered to be statistically significant. The results obtained are shown in FIG. 2. As shown in FIG. 2, scratching behaviors at 20 minutes or later after CP4715 administration were significantly suppressed (P<0.05), and this effect was sustained until 60 minutes after administration.

In Vivo Extracellular Recording from the Posterior Horn Surface of the Spinal Cord in FADS Mice

FADS mice were administered intraperitoneally with 1.5 g/kg of urethane and anesthetized, followed by dorsal lumbar laminectomy. The first to sixth lumbar vertebrae were exposed, and each mouse was set on a stereotaxic apparatus. Under a microscope, the dural, arachnoidal and pial membranes were removed, and the spinal cord surface was warmed at 36±1 °C, followed by perfusion at 15 to 20 mL/min with an artificial cerebrospinal fluid (composition: NaCl (117 mM), KCl (3.6 mM), CaCl₂) (2.5 mM), MgCl₂ (1.2 mM), NaH₂PO₄ (1.2 mM), glucose (11 mM), NaHCO₃ (25 mM)) saturated with 95% 02 to 5% CO₂. Into the exposed spinal cord, a tungsten electrode (tip diameter: 25 mm, tip impedance: 9 to 12 MΩ) was inserted (latero-medial), and records were obtained from neurons in the posterior horn surface of the spinal cord.

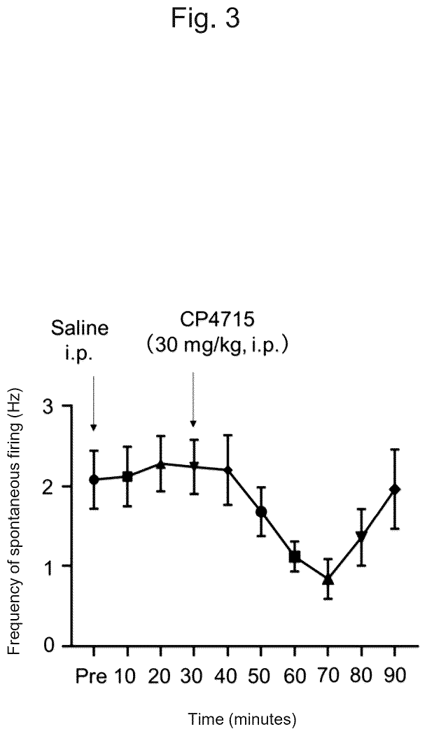

CP4715 was dissolved at 89.5 mM in a diluent (physiological saline containing 50% DMSO and 89.5 mM hydrochloric acid), from which an amount corresponding to 53.7 μmol/kg per mouse was then taken and diluted with 0.3 mL of physiological saline, and then administered intraperitoneally (i.p.). 0.3 mL of physiological saline containing an equal amount of the diluent as calculated based on the body weight was administered as a control. When recorded from the spinal cord posterior horn of the FADS mice, neurons with spontaneous firing could be recorded. When the above control solution was first administered intraperitoneally, there was little change in the frequency of spontaneous firing during 30 minutes after administration. When CP4715 was subsequently administered intraperitoneally, the frequency of firing was started to decrease from 20 minutes after administration, and the frequency of firing was most strongly suppressed at the time point of 40 minutes after administration and then returned to the initial frequency of firing at 60 minutes after administration (FIG. 3).

These results suggest that periostin directly acts on sensory neuron cells to induce neuron firing, and that CP4715 inhibits binding between periostin and sensory neuron cells to thereby suppress neuron firing, as a result of which scratching behaviors are suppressed. The fact that periostin causes scratching behaviors in FADS mice is evident from the finding that scratching behaviors are suppressed upon administration of an anti-periostin monoclonal antibody.

### Example 2

Inhibition of Scratching Behaviors in FADS Mice Upon Single Systemic Administration of αvβ3 Integrin Inhibitors

The following compounds reported as αvβ3 integrin inhibitors were used and compared for their inhibitory effect against scratching behaviors in FADS mice. Namely, the compounds used were CP derivative 2, CP derivative 6, CP derivative 7 and CP derivative 9, each having a skeletal structure in common with CP4715, as well as SC-68448, BS-1417, SB-273005, GLPG-0187, Cilengitide, SM-256 and SB-267268. Moreover, SB-267268 enantiomer was used as a negative control. These compounds are all known compounds. CP4715, CP derivative 2, CP derivative 6, CP derivative 7 and CP derivative 9 are disclosed in Nanri Y, et al. Am J Respir Cell Mol Biol. 62:204-216, 2020. Likewise, SC-68448 is disclosed in Carron C P, et al. Cancer Res. 58:1930-1935, 1998, BS-1417 is disclosed in Sakai M, Yoshimura R. Biol Pharm Bull. 44:416-421, 2021, SB-273005 is disclosed in Badger A M, et al. Arthritis Rheum. 44:128-137, 2001, GLPG-0187 is disclosed in Helen M, et al. J Med. Chem. 57:6301-6315, 2014, Cilengitide is disclosed in Eskens F A, et al. Eur J Cancer. 39:917-926, 2003, SM-256 is disclosed in Mousa S A, et al. J Cardiovasc Pharmacol. 33:641-646, 1999, and SB-267268 is disclosed in Wilkinson-Berka J L, et al. Invest Ophthalmol Vis Sci. 47:1600-1605, 2006.

The chemical name (free form), chemical structure and CAS registration number (free form) of each compound are as shown below. CP4715, CP derivative 6 and SC-68448 are as described above.

CP derivative 2: (S)-3-(3-fluoro-4-(4-((1,4,5,6-tetrahydropyrimidin-2-yl)amino)piperidin-1-yl)benzamido)-2-(phenylsulfonamido)propanoic acid (CAS registration number: 247034-18-0)

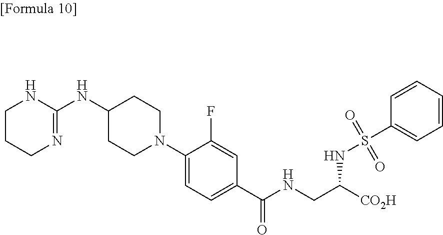 [SMILES: O=C(NC[C@H](NS(=O)(=O)c1ccccc1)C(=O)O)c1ccc(N2CCC(NC3=NCCCN3)CC2)c(F)c1]

CP derivative 7: (S)-3-(2-fluoro-4-((S)-3-((1,4,5,6-tetrahydropyrimidin-2-yl)amino)piperidin-1-yl)benzamido)-2-(phenylsulfonamido)propanoic acid (CAS registration number: 334619-09-9)

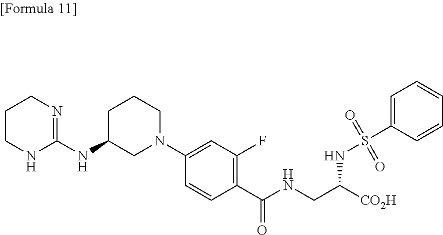 [SMILES: O=C(NC[C@H](NS(=O)(=O)c1ccccc1)C(=O)O)c1ccc(N2CCC[C@H](NC3=NCCCN3)C2)cc1F]

CP derivative 9: (S)-3-(4-((S)-3-((1,4,5,6-tetrahydropyrimidin-2-yl)amino)piperidin-1-yl)benzamido)-2-(thiophene-2-sulfonamido)propanoic acid (CAS registration number: 334619-69-1)

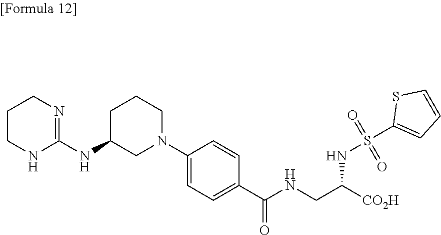 [SMILES: O=C(NC[C@H](NS(=O)(=O)c1cccs1)C(=O)O)c1ccc(N2CCC[C@H](NC3=NCCCN3)C2)cc1]

BS-1417: (2S)-5-(4-(3-((5-fluoro-1,4,5,6-tetrahydropyrimidin-2-yl)amino)phenyl)piperazin-1-yl)-2-((isobutoxycarbonyl)amino)-5-oxopentanoic acid (CAS registration number: 461719-50-6)

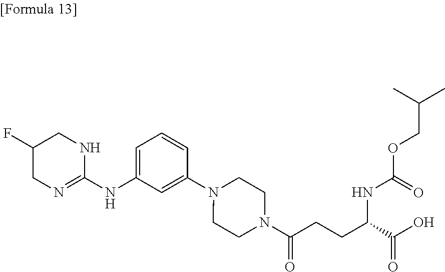 [SMILES: CC(C)COC(=O)N[C@@H](CCC(=O)N1CCN(c2cccc(NC3=NCC(F)CN3)c2)CC1)C(=O)O]

SB-273005: (S)-2-(8-(2-(6-(methylamino)pyridin-2-yl)ethoxy)-3-oxo-2-(2,2,2-trifluoroethyl)-2,3,4,5-tetrahydro-1H-benzo[c]azepin-4-yl)acetic acid (CAS registration number: 205678-31-5)

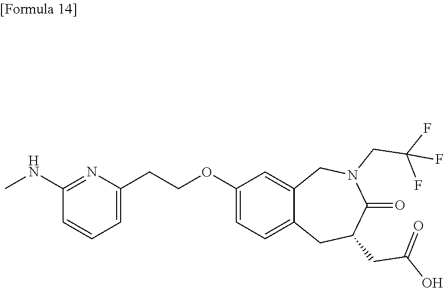 [SMILES: CNc1cccc(CCOc2ccc3c(c2)CN(CC(F)(F)F)C(=O)[C@H](CC(=O)O)C3)n1]

GLPG-0187: (S)-3-((2,5-dimethyl-6-(4-(5,6,7,8-tetrahydro-1,8-naphthyridin-2-yl)piperidin-1-yl)pyrimidin-4-yl)amino)-2-((4-methoxyphenyl)sulfonamido)propanoic acid (CAS registration number: 1320346-97-1)

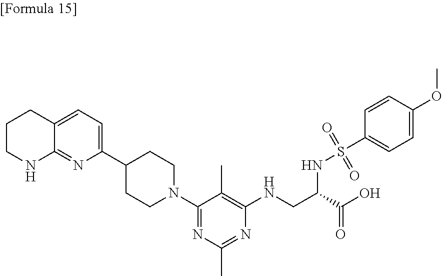 [SMILES: COc1ccc(S(=O)(=O)N[C@@H](CNc2nc(C)nc(N3CCC(c4ccc5c(n4)NCCC5)CC3)c2C)C(=O)O)cc1]

Cilengitide: 2-((2S,5R,8S,11S)-5-benzyl-11-(3-guanidinopropyl)-8-isopropyl-7-methyl-3,6,9,12,15-pentaoxo-1,4,7,10,13-pentaazacyclopentadecan-2-yl)acetic acid (CAS registration number: 188968-51-6)

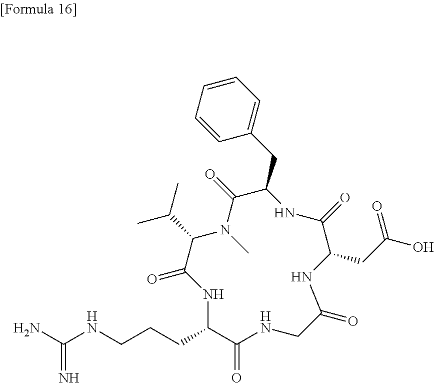 [SMILES: CC(C)[C@H]1C(=O)N[C@@H](CCCNC(=N)N)C(=O)NCC(=O)N[C@@H](CC(=O)O)C(=O)N[C@H](Cc2ccccc2)C(=O)N1C]

SM-256: (S)-3-(1-(3-((1H-imidazol-2-yl)amino)propyl)-1H-indazole-5-carboxamido)-2-((2,4,6-trimethylphenyl)sulfonamido)propanoic acid (CAS registration number: 192944-48-2)

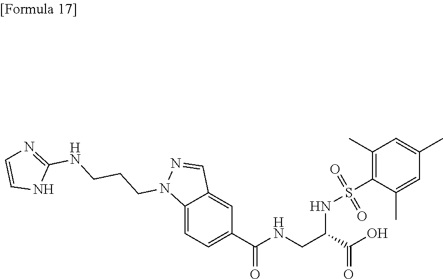 [SMILES: Cc1cc(C)c(S(=O)(=O)N[C@@H](CNC(=O)c2ccc3c(cnn3CCCNc3ncc[nH]3)c2)C(=O)O)c(C)c1]

SB-267268: (S)-2-(3-oxo-8-(3-(pyridin-2-ylamino)propoxy)-2-(2,2,2-trifluoroethyl)-2,3,4,5-tetrahydro-1H-benzo[c]azepin-4-yl)acetic acid (CAS registration number: 205678-26-8)

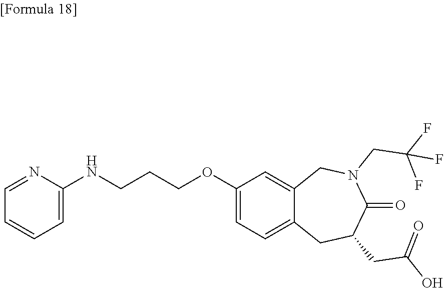 [SMILES: O=C(O)C[C@@H]1Cc2ccc(OCCCNc3ccccn3)cc2CN(CC(F)(F)F)C1=O]

SB-267268 enantiomer: (R)-2-(3-oxo-8-(3-(pyridin-2-ylamino)propoxy)-2-(2,2,2-trifluoroethyl)-2,3,4,5-tetrahydro-1H-benzo[c]azepin-4-yl)acetic acid (CAS registration number: 205678-27-9)

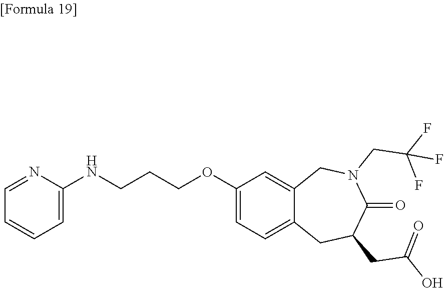 [SMILES: O=C(O)C[C@H]1Cc2ccc(OCCCNc3ccccn3)cc2CN(CC(F)(F)F)C1=O]

CP4715 was provided by Meiji Seika Pharma Co., Ltd., Japan. Cilengitide and SB-273005 were purchased from Selleck. GLPG-0187 was purchased from MedChemExpress. CP4715 and CP derivative 2 can be prepared according to the procedures described in WO1999/052872. CP derivative 6, CP derivative 7 and CP derivative 9 can be prepared according to the procedures described in WO2001/027082. Cilengitide can be prepared according to the procedures described in DE19534177. GLPG-0187 can be prepared according to the procedures described in WO2011/119282. SC-68448 can be prepared according to the procedures described in WO97/08145. BS-1417 can be prepared according to the procedures described in WO2002/074743. SB-273005 can be prepared according to the procedures described in WO98/14192. SM-256 can be prepared according to the procedures described in WO09/723480. SB-267268 and SB-267268 enantiomer can be prepared according to the procedures described in WO98/14192.

Prior to the administration of the above αvβ3 integrin inhibitors, FADS mice were recorded for 60 minutes with a video camera and then allowed to rest for 15 minutes. The compounds except for GLPG-0187 were each dissolved at 89.5 mM in a diluent (physiological saline containing 50% DMSO and 89.5 mM HCL), from which an amount corresponding to 53.7 μmol/kg per mouse was then taken and diluted with 0.3 mL of PBS, and then administered intraperitoneally. Due to its low solubility, GLPG-0187 was dissolved at 8.95 mM in the diluent, from which an amount corresponding to 5.37 μmol/kg per mouse was then taken and diluted with 0.3 mL of PBS, and then administered intraperitoneally. Mice administered with 0.3 mL of PBS containing an equal amount of the diluent as calculated based on the body weight were used as a control group.

The mice were recorded for 60 minutes with a video camera immediately after administration of the above αvβ3 integrin inhibitors. The number of scratchings before administration was used as a baseline, and % change from the baseline after administration was calculated for each compound. For statistical analysis, paired t-test was used. A P-value of less than 0.05 was considered to be statistically significant. The effect of an inhibitor whose mean change from the baseline was −65% or more at a P-value of less than 0.01 was expressed as “++”; the effect of an inhibitor whose mean change from the baseline was −60% or less at a P-value of less than 0.05 was expressed as “+”; and the effect of an inhibitor whose mean change from the baseline was −60% or less at a P-value of 0.05 or higher was expressed as “−.”

Table 1 shows the % change from the baseline in the number of scratchings for each compound. Except for GLPG-0187, the dose of each compound was equally set to 53.7 μmol/kg.

TABLE 1

| | | % Change from baseline |
|---|---|---|
| Compound name | n, dose | (mean ± standard error) |
| CP4715 | (n = 10) 53.7 μmol/kg | −68.0 ± 14.0 |
| SC-68448 | (n = 4) 53.7 μmol/kg | −83.8 ± 7.7 |
| CP derivative 6 | (n = 4) 53.7 μmol/kg | −77.8 ± 7.9 |
| BS-1417 | (n = 4) 53.7 μmol/kg | −43.3 ± 11.9 |
| SB-273005 | (n = 5) 53.7 μmol/kg | −33.0 ± 11.6 |
| CP derivative 7 | (n = 4) 53.7 μmol/kg | −52.0 ± 26.6 |
| CP derivative 2 | (n = 4) 53.7 μmol/kg | −30.2 ± 18.6 |
| CP derivative 9 | (n = 4) 53.7 μmol/kg | −24.5 ± 19.9 |
| GLPG-0187 | (n = 4) 5.37 μmol/kg | −38.9 ± 31.2 |
| Cilengitide | (n = 5) 53.7 μmol/kg | −31.7 ± 17.8 |
| SM-256 | (n = 4) 53.7 μmol/kg | −22.5 ± 19.6 |
| SB-267268 | (n = 4) 53.7 μmol/kg | −5.5 ± 13.4 |
| SB-267268 enantiomer | (n = 4) 53.7 μmol/kg | +45.5 ± 28.8 |
| Vehicle | (n = 8) | −17.6 ± 14.3 |

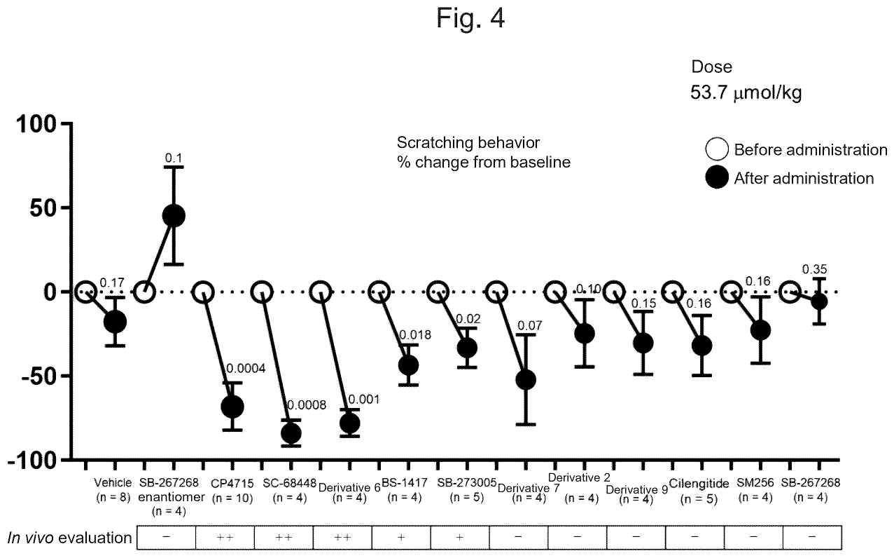

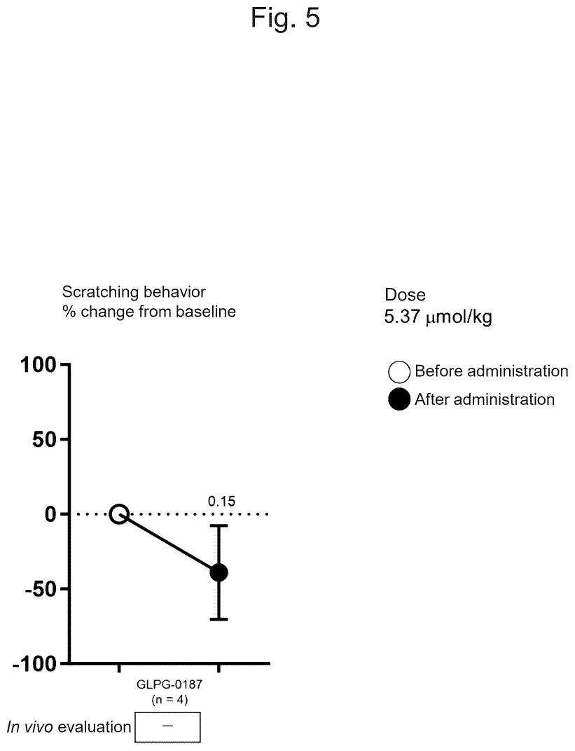

The results obtained for the compounds except for GLPG-0187 are shown in FIG. 4, while the results obtained for GLPG-0187 are shown in FIG. 5. The numerical values shown inside FIGS. 4 and 5 represent P-values. As can be seen from Table 1 and FIG. 4, the % change from the baseline in the number of scratchings was “++” for CP4715, SC-68448 and CP derivative 6, whereas it was “+” for BS-1417 and SB-273005, and “−” for the other compounds.

Inhibitory Effect of αvβ Integrin Inhibitors Against Periostin-Dependent Cell Adhesion

For use in cell adhesion experiments, SW480 cells, a human colon cancer cell line not expressing integrin β3, were genetically engineered to stably express human integrin β3 (Nanri Y, et al. Am J Respir Cell Mol Biol. 62:204-216, 2020). The compounds shown in Table 1 were examined for their inhibitory activity against adhesion between integrin αvβ3 and periostin according to the following procedures. First, a 96-well plate was treated with 10 μg/mL recombinant human periostin (Bio-Techne) at 4 °C for 16 hours to immobilize periostin. The SW480 cells expressing integrin αvβ3 (5×10⁴ cells) were reacted at room temperature for 15 minutes with the above compounds at 0.001, 0.01, 0.1 and 1 μM in serum-free DMEM (Dulbecco's Modified Eagle Medium) containing 0.5% bovine serum albumin and 0.25 mM MnCl₂. A group receiving no compound was used as a control. The reacted cell suspensions were added to separate wells of the above human periostin-immobilized plate, and the plate was centrifuged at 10×g for 5 minutes and then cultured at 37 °C for 60 minutes. Then, after the adhered cells were washed to remove unbound cells, the adhered cells were fixed with 1% formaldehyde and stained with 0.5% crystal violet. The fixed and stained cells were solubilized with 0.2% Triton X-100 and measured for absorbance at 595 nm. The absorbance of the control (i.e., the group receiving no compound) was set to 0% inhibition. Likewise, the same procedures were repeated to measure the absorbance of crystal violet derived from the cells adhered to a bovine albumin-immobilized plate in the absence of the compounds, and this absorbance was set to 100% inhibition. Cell adhesion in this test system is periostin-dependent, and a compound inhibiting this cell adhesion can be referred to as a periostin receptor antagonist.

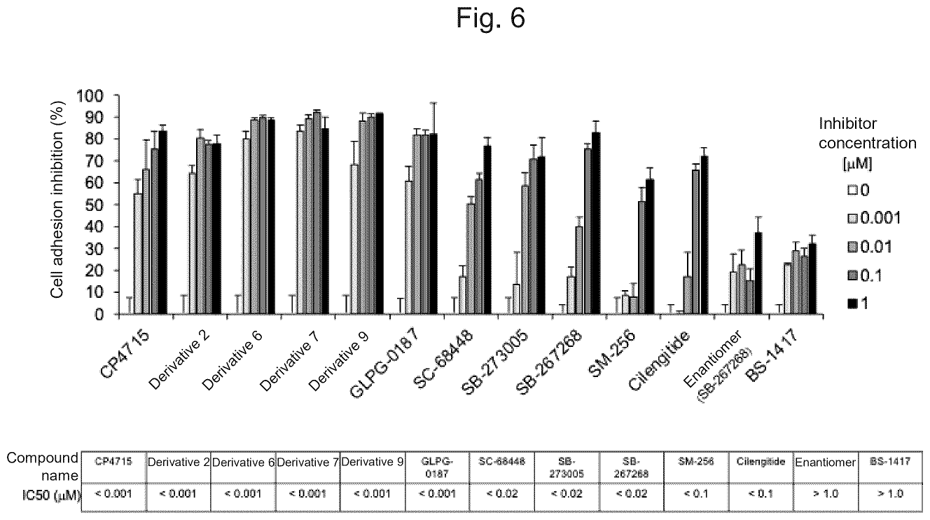

The results obtained are shown in FIG. 6. The 50% inhibition concentration (IC₅₀ value) for cell adhesion inhibitory activity was 0.001 μM or less in CP4715, CP derivative 2, CP derivative 6, CP derivative 7, CP derivative 9 and GLPG-0187, and these compounds showed a high cell adhesion inhibitory effect. The IC₅₀ values of SC-68448, SB-273005 and SB-267268 were each 0.02 μM or less, and these compounds showed a moderate cell adhesion inhibitory effect. The IC₅₀ values of SM-256 and Cilengitide were each 0.1 μM or less, and their inhibitory effect was low. Moreover, the IC₅₀ values of SB-267268 enantiomer and BS-1417 were each 1 μM or more.

In the above compounds, their inhibitory effect against cell adhesion and their inhibitory effect against scratching behaviors in FADS mice were found not to always correlate with each other. CP4715, CP derivative 2, CP derivative 6, CP derivative 7 and CP derivative 9 all showed strong cell adhesion inhibitory activity with an IC₅₀ value of 0.001 μM or less as periostin receptor antagonists. On the other hand, in terms of their inhibitory effect against scratching behaviors in FADS mice, CP4715 and CP derivative 6 exerted a strong inhibitory effect expressed as “++” whereas CP derivative 2, CP derivative 7 and CP derivative 9 showed only a weak effect expressed as “−.” SC-68448 showed a strong inhibitory effect expressed as “++” against scratching behaviors in FADS mice, whereas SB-273005 and SB-267268, each having the same cell adhesion inhibitory activity as SC-68448, were weak in their inhibitory effect against scratching behaviors, expressed as “+” and “−” respectively.

### Example 3

### Inhibition of Scratching Behaviors in FADS Mice Upon Repeated Percutaneous Administration of CP4715

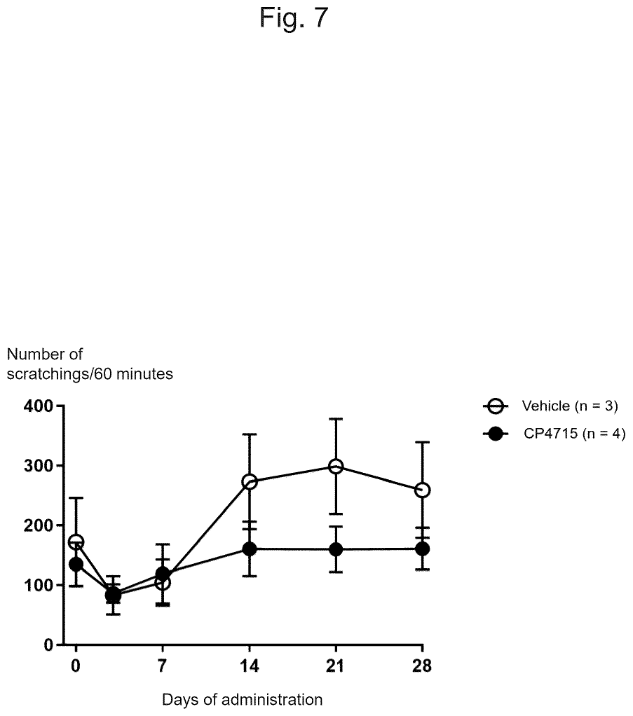

CP4715 was dissolved in a diluent (5% DMSO/15% N-methyl-2-pyrrolidone/80% ethanol) to prepare a 26.85 mM solution. To facial rash lesions in FADS mice, 30 μL of the CP4715 solution was externally applied once a day for 28 consecutive days. Mice administered percutaneously with an equal volume of the diluent were used as a control group. Before administration and on Days 3, 7, 14, 21 and 28 of administration, the number of scratchings during 60 minutes was counted for evaluation. The results obtained are shown in FIG. 7. As shown in FIG. 7, the time-dependent enhancement of scratching behaviors in FADS mice was suppressed upon external application of CP4715.

### INDUSTRIAL APPLICABILITY

The present invention enables the provision of not only a prophylactic or therapeutic agent for a periostin-mediated disease and pruritus associated with the disease, which is effective in the improvement of the disease and the pruritus, but also a pharmaceutical composition for preventing or treating the disease and the pruritus, and a method for preventing or treating the disease and the pruritus, etc.

1. A prophylactic or therapeutic agent for a periostin-mediated disease or pruritus associated with the disease, which comprises a periostin receptor antagonist as an active ingredient, wherein the antagonist is at least one compound selected from the group consisting of a compound of the following formula (I): 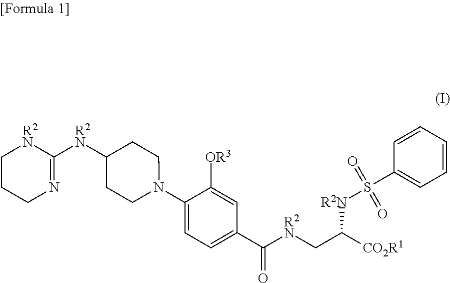
[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid and (S)-2-(phenylsulfonamido)-3-(4-((S)-3-((1,4,5,6-tetrahydropyrimidin-2-yl)amino)piperidin-1-yl)benzamido)propanoic acid, or a prodrug thereof, or a pharmacologically acceptable salt thereof, or a hydrate or solvate thereof.

2. The prophylactic or therapeutic agent according to claim 1, wherein the compound of formula (I) is (S)-3-(3-methoxy-4-(4-((1,4,5,6-tetrahydropyrimidin-2-yl)amino)piperidin-1-yl)benzamido)-2-(phenylsulfonamido)propanoic acid.

3. The prophylactic or therapeutic agent according to claim 1, wherein the periostin-mediated disease is atopic dermatitis, prurigo nodularis, bullous pemphigoid, stasis dermatitis, cutaneous T-cell lymphoma, systemic scleroderma, a keloid or hypertrophic scar, allergic rhinitis, a chronic liver disease or a chronic kidney disease.

4. The prophylactic or therapeutic agent according to claim 1, wherein the periostin-mediated disease is atopic dermatitis.

5. A pharmaceutical composition for preventing or treating a periostin-mediated disease or pruritus associated with the disease, which comprises a periostin receptor antagonist as an active ingredient, wherein the antagonist is at least one compound selected from the group consisting of a compound of the following formula (I): 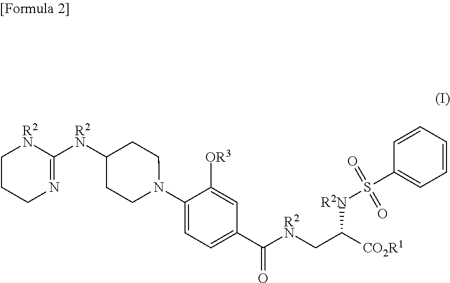
[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid and (S)-2-(phenylsulfonamido)-3-(4-((S)-3-((1,4,5,6-tetrahydropyrimidin-2-yl)amino)piperidin-1-yl)benzamido)propanoic acid, or a prodrug thereof, or a pharmacologically acceptable salt thereof, or a hydrate or solvate thereof.

6. The pharmaceutical composition according to claim 5, wherein the compound of formula (I) is (S)-3-(3-methoxy-4-(4-((1,4,5,6-tetrahydropyrimidin-2-yl)amino)piperidin-1-yl)benzamido)-2-(phenylsulfonamido)propanoic acid.

7. The pharmaceutical composition according to claim 5, wherein the periostin-mediated disease is atopic dermatitis, prurigo nodularis, bullous pemphigoid, stasis dermatitis, cutaneous T-cell lymphoma, systemic scleroderma, a keloid or hypertrophic scar, allergic rhinitis, a chronic liver disease or a chronic kidney disease.

8. The pharmaceutical composition according to claim 5, wherein the periostin-mediated disease is atopic dermatitis.

9. A use of at least one compound selected from the group consisting of a compound of the following formula (I): 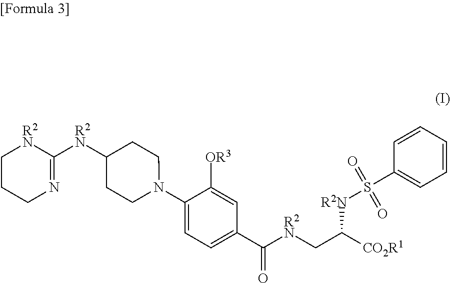
[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid and (S)-2-(phenylsulfonamido)-3-(4-((S)-3-((1,4,5,6-tetrahydropyrimidin-2-yl)amino)piperidin-1-yl)benzamido)propanoic acid, or a prodrug thereof, or a pharmacologically acceptable salt thereof, or a hydrate or solvate thereof for the manufacture of a prophylactic or therapeutic agent for a periostin-mediated disease or pruritus associated with the disease.

10. The use according to claim 9, wherein the compound of formula (I) is (S)-3-(3-methoxy-4-(4-((1,4,5,6-tetrahydropyrimidin-2-yl)amino)piperidin-1-yl)benzamido)-2-(phenylsulfonamido)propanoic acid.

11. The use according to claim 9, wherein the periostin-mediated disease is atopic dermatitis, prurigo nodularis, bullous pemphigoid, stasis dermatitis, cutaneous T-cell lymphoma, systemic scleroderma, a keloid or hypertrophic scar, allergic rhinitis, a chronic liver disease or a chronic kidney disease.

12. The use according to claim 9, wherein the periostin-mediated disease is atopic dermatitis.

13. A method for preventing or treating a periostin-mediated disease or pruritus associated with the disease, which comprises administering an effective amount of a periostin receptor antagonist to a subject in need of treatment, wherein the antagonist is at least one compound selected from the group consisting of a compound of the following formula (I): 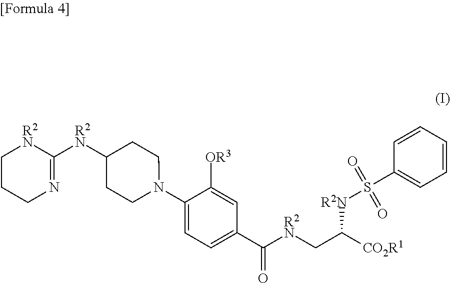
[wherein
R¹ is a hydrogen atom, an alkyl group, an aryl group or an aralkyl group,
each R² is independently a hydrogen atom, an alkyl group, an aryl group or an aralkyl group, and
R³ is an alkyl group, an aryl group or an aralkyl group], 3-(3,5-dichlorophenyl)-3-(2-(3-guanidinobenzamido)acetamido)propanoic acid and (S)-2-(phenylsulfonamido)-3-(4-((S)-3-((1,4,5,6-tetrahydropyrimidin-2-yl)amino)piperidin-1-yl)benzamido)propanoic acid, or a prodrug thereof, or a pharmacologically acceptable salt thereof, or a hydrate or solvate thereof.

14. The method according to claim 13, wherein the compound of formula (I) is (S)-3-(3-methoxy-4-(4-((1,4,5,6-tetrahydropyrimidin-2-yl)amino)piperidin-1-yl)benzamido)-2-(phenylsulfonamido)propanoic acid.

15. The method according to claim 13, wherein the periostin-mediated disease is atopic dermatitis, prurigo nodularis, bullous pemphigoid, stasis dermatitis, cutaneous T-cell lymphoma, systemic scleroderma, a keloid or hypertrophic scar, allergic rhinitis, a chronic liver disease or a chronic kidney disease.

16. The method according to claim 13, wherein the periostin-mediated disease is atopic dermatitis.
